(19) United States
(12) Patent Application Publication
(10) Pub. No.: US 2016/0018373 A1
(43) Pub. Date: Jan. 21, 2016

(54) SYSTEMS AND METHODS FOR MONITORING AND CONTROLLED CAPTURE OF AIR SAMPLES FOR ANALYSIS

(71) Applicants: ODOTECH INC., Montreal (CA); TOTAL S.A., Courbevoie (FR)

(72) Inventors: Thierry PAGÉ, Montréal (CA); Nikolaos Alexandros VERIOTES, Calgary (CA)

(73) Assignees: TOTAL S.A., Courbevoie (FR); ODOTECH INC., Montreal, QC (CA)

(21) Appl. No.: 14/774,880

(22) PCT Filed: Mar. 12, 2014
(86) PCT No.: PCT/CA2014/000227

§ 371 Date: Sep. 11, 2015

Related U.S. Application Data
(60) Provisional application No. 61/784,654, filed on Mar. 14, 2013.

(51) Int. Cl. G01N 33/00; G01N 1/22

(52) U.S. Cl. CPC G01N 33/0032; G01N 1/2273

(57) ABSTRACT

There are provided methods for analyzing air including monitoring the air. For example, monitoring the air includes electronically sensing the air and determining whether an event in the air is occurring based on at least the electronically sensing. If an event is occurring, a sample of the air is collected for a further analysis. For example, the analysis is carried out to identify components present in the sample. There are also provided systems for analyzing air comprising : an air intake; an electronic sensor and a controller configured to monitor the air. Monitoring air includes controlling the air intake, controlling the electronic sensor to electronically sensing the air and determining whether an event in the air is occurring based on at least the electronically sensing. If an event is occurring, a sample of the air is collected for further analysis (e.g. to identify components present in the sample).

### CROSS-REFERENCE TO RELATED APPLICATIONS

The present application claims priority on U.S. 61/784,654 filed on Mar. 14, 2013, that is hereby incorporated by reference in its entirety.

### FIELD OF THE DISCLOSURE

The present subject-matter relates to monitoring of air, and more particularly to monitoring the air to trigger a collection of a sample of the air for a further analysis.

### BACKGROUND OF THE DISCLOSURE

Monitoring air quality is important in many situations and knowing what to monitor and trigger the monitoring or sampling event adds a layer of complexity. Poor air quality having toxic or odorous chemicals can result in stress effects resulting in detrimental health or environmental effects, or in the least cause a nuisance. Monitoring air quality is particularly important in areas that are near sources of emissions, such as industrial operations, municipal activities, airports, port or mining operations.

Current continuous air quality analyzers are limited in the number of chemical compounds that may be analyzed. One analyzer cannot detect an array of chemicals; a specific analyzer is required for each suspected chemical component. Furthermore; these analyzers are expensive and need to be installed in a controlled environment. The equipment requires regular calibration and maintenance to ensure optimal performance. These analyzers also need to be ranged properly to but as the maximum range increases the detection at the lower end of the range is compromised. Higher ranging instruments typically are at the expense of lower detection limits. The opposite is true for sensitive ranging, low detection ranging resulting in loss of higher range readings or off scale measurement.

Analyzers that have lower detection thresholds and capable of measuring large ranges of chemicals are non-continuous. A gas chromatography-mass spectrometry is one example of such analyzer. Where poor air quality events are intermittent and of a short duration, a non-continuous analyzer may miss these events.

### SUMMARY OF THE DISCLOSURE

It would thus be highly desirable to be provided with a system or method that would at least partially address the disadvantages of the existing technologies.

According to one aspect, there is provided a method for analyzing air including monitoring the air. Monitoring the air includes electronically sensing the air and determining whether an event in the air is occurring based on at least the electronically sensing. If an event is occurring, a sample of the air is collected for a further analysis. For example, the analysis may be carried out to identify components present in the sample.

According to another aspect, there is provided a method for analyzing air without the need to know the exact chemicals in the air. The method can uses at least two or a multiple of sensors to monitor the air quality. The sensing unit can continuously monitor the air electronically to determine if an event is occurring based on the electronic dimensional array signature. Each sensor can respond uniquely and the multiple sensors together generate a unique and distinctive signature. If an event is occurring, a sample of the air can be collected for a further analysis. The unique electronic array signature can then be correlated to the chemical analysis developing a database of responses to chemical composition with a high level of confidence.

According to another aspect, there is provided a system for analyzing air. The system includes an air intake, an electronic sensor and a controller. The controller is configured to monitor the air. Monitoring the air includes controlling the air intake, controlling the electronic sensor to electronically sensing the air and determining whether an event in the air is occurring based on at least the electronically sensing. If an event is occurring, a sample of the air is collected for a further analysis. For example, the analysis may be carried out to identify components present in the sample.

According to another aspect, there is provided a system for analyzing air quality. The system includes an air intake, a multi electronic sensors and a controller. The controller is configured to monitor the air quality. Monitoring the air quality includes controlling the air intake, controlling the electronic sensors to electronically sense the air quality and determining whether an event in the air is occurring based the cumulative electronic signature responses from the sensors. If an event is occurring, the predefined response threshold triggers a sample of the air to be collected for further chemical or physical analysis or instantaneous analysis depending on the equipment. For example, samples may be triggered based one or more parameters such as a specific array signature, and/or an electronic threshold limit trigger exceedance and/or meteorological data such as wind vectoring to collect and identify components present in the sample.

### BRIEF DESCRIPTION OF THE DRAWINGS

The following drawings represent non-limitative examples in which:

FIG. 1 is a schematic diagram of an event detection system in accordance to one exemplary embodiment;

FIG. 2 is a schematic diagram of a combined event detection and gas analysis stem in accordance with one exemplary embodiment;

FIG. 3 is a schematic diagram of an exemplary method for monitoring the air;

FIG. 4 is a schematic diagram of another exemplary method for monitoring the air;

FIG. 5 is a schematic diagram of an exemplary method for carrying out a collection of the sample of the air quality for further analysis;

FIG. 6 is a schematic diagram of an exemplary method for calculating the emission rate at an emission source; and

FIG. 7 is a schematic diagram of an exemplary method for validating dispersion modeling used to predict concentration levels of specified gases.

### DETAILED DESCRIPTION OF VARIOUS EMBODIMENTS

The following examples are presented in a non-limiting manner.

The term “event” as used herein when describing electronically sensing air refers to a result obtained when a change in the chemical composition of the air is detected or when a predetermined electronic sensory response or a predetermined electronic dimensional array signature (EDAS) is detected or not. For example, the change in the chemical composition of the air can be a change of the concentration of at least one compound. For example, the change in the chemical composition of the air can be a change in the proportion that at least two compounds have with respect to one another in the air. For example, such a change can be a detected change representing a value that is greater than a predetermined threshold value for a given compound. For example, such a change can be a change in the pattern of the data received when electronically sensing the air. For example, such a change can be a result obtained when a compound or a class of compounds is present or not in the air and for example at a concentration higher than a predetermined threshold value. For example, the expressions “electronic sensory response” and “electronic dimensional array signature” (EDAS) can be used when describing electronically sensing air quality that refers to a result obtained when a change in the chemical composition of the air quality is detected by the multitude of sensors working together to providing a unique electrical signature response.

For example, the air to be analyzed by the methods and systems of the present disclosure can be air found in various types of places. For example, it can applicable to indoor and well as outdoor air quality monitoring. The unit(s) can be in close proximity of an emission source; the unit(s) can also be deployed remote from the source surrounding an area of interest such as a small community; the units can be distributed over large area and complex terrain; the unit(s) can also be air found in a duct or a conduit. For example, the air can be disposed at a location such that it can be contacting at least one sensor of a system as defined in the present disclosure or at least one sensor used for carrying out a method as defined in the present disclosure. For example, the quantity or volume of air to be analyzed for the monitoring and/or sensing can be as low as the lowest quantity of air to be necessary for a given type of sensor. For example, in the methods and systems of the presen disclosure a single sensor or a plurality of sensors can be used. For example, different sensor technologies can be used such as MOS (Metal-Oxide Semiconductor), QMB (Quartz Microbalance), IRS (Infra-Red Sensor), CPS (Conducting Polymer Sensor), SAW (Surface Acoustic Wave), OFS (Optical Fiber Sensor), and others. These sensor types have different sensitivity, selectivity, robustness and service life characteristics. The choice and combination of technologies depends primarily on the type of application. Odorous molecule recognition and/or quantification can made indirectly by measuring changes in some physical properties of the sensors, such as electrical conductivity and the resonance frequency. For example, such sensors can be used in electronic noses.

For example, samples may be triggered based one or more parameters such as a specific array signature, and/or an electronic threshold limit trigger excedance and/or meteorological data such as wind vectoring to collect and identify components present in the sample.

For example, each sensor can react in a specific way to one or more chemical components in the air enabling a unique EDAS. The methods and systems of the present disclosure can incorporate a multitude of sensors that on their own provide little information but as a collective have the ability to provide an electronic dimensional array signature of the chemical components in the air.

For example, the methods can further comprise carrying out the further analysis of the sample for identifying at least the major components present in the sample.

For example, the methods can further comprise carrying out the further analysis, wherein the further analysis is a chemical analysis of the sample for identifying components present in the sample.

For example, the methods can further comprise carrying out the further analysis, wherein the further analysis is a chemical analysis of the sample for identifying at least one component present in the sample.

For example, the further analysis can be carried out by means of a non-continuous apparatus.

For example, the further analysis is carried out by means of an olfactometer, a GC-MS (Gas Chromatography-Mass Spectroscopy), a SPME (Solid-Phase Micro-Extraction), a PFPD (Pulse-Flame Photometric Detectors), flame photometric detectors, flame ionization detector, a tandem mass spectrometry, gas chromatography-mass spectrometry—olfactory port, Photoluminescence-based detector, fourier transform infrared spectroscopy or combinations thereof.

For example, the monitoring of the air can be a continuous monitoring.

For example, the monitoring the air can be a continuous qualitative monitoring.

For example, the electronic sensing can be a qualitative evaluation of the air.

For example, the electronically sensing of the air and the determination of whether an event is occurring can be carried out substantially in real time.

For example, the electronically sensing of the air and the determination of whether an event is occurring can be repeated at short time intervals apart, and wherein the monitoring of the air is substantially continuous.

For example, a result obtained from the electronically sensing of the air can be compared to a predefined sensor reaction pattern, a signature pattern or sensor fingerprints in order to determine if an event is occurring.

For example, the monitoring can further comprise conditioning a volume of the air to improve sensing accuracy and wherein electronically sensing the air includes sensing the conditioned volume of the air.

For example, conditioning the volume of the air can comprise adjusting at least one of the temperature or humidity of the sample.

For example, wherein the determination of whether an event is occurring can be further based on at least one of at least one weather characteristic of the air; and at least one previous result of the electronic sensing.

For example, the at least one weather characteristic can be selected from temperature, humidity, pressure, wind direction, wind speed, and solar radiation.

For example, collecting the sample for further analysis can be made in accordance with parameters that are adjusted in view of the result obtained from the electronically sensing of the air that is compared to a predefined sensor reaction pattern, a signature pattern or sensor fingerprints.

For example, the parameters can comprise at least two parameters chosen from time, period, frequency, temperature and level of humidity.

For example, determining whether an event in the air is occurring can be based on whether a result of the electronic sensing satisfies at least one predefined condition.

For example, the electronic sensing can be carried out using a multi-sensor array based apparatus, wherein at least one of the sensors of the apparatus is non-specific.

For example, the electronic sensing can be carried out using an electronic nose.

For example, a plurality of parameters of the analysis of the sample can be selected based at least on the result of the electronic sensing.

For example, the further analysis of the sample of air can comprises conducting a gas chromatography of the air.

For example, the methods can further comprise:

after triggering the start of the collecting of the sample of the air, if the event is no longer occurring, triggering an end of the collecting of the sample of the air.

For example, the method can further comprise:

after triggering the start of the collecting of a sample of the air, monitoring a volume of the collected sample of the air within the receptacle; if the receptacle becomes substantially full, ending the collecting of the sample of the air.

For example, the electronically sensing the air; and determining whether an event in the air is occurring based on at least the electronically sensing can be carried out by means of a predetermined electronic sensory response and presence of such a predetermined electronic sensory response confirms occurrence of the event.

For example, the electronically sensing the air; and determining whether an event in the air is occurring based on at least the electronically sensing can be carried out by noting a change in the chemical composition of the air is detected or when a predetermined electronic sensory response or a predetermined electronic dimensional array signature (EDAS) is detected or not.

For example, the electronically sensing the air; and determining whether an event in the air is occurring based on at least the electronically sensing can be carried out by means of a predetermined electronic dimensional array signature and presence of such a predetermined electronic dimensional array signature in the air confirms occurrence of the event.

For example, the monitoring of the air can be a continuous qualitative monitoring.

For example, the result obtained from the electronically sensing of the air can be compared to a predefined sensor reaction pattern, a signature pattern or sensor fingerprints in order to determine if an event is occurring.

For example, controlling the air intake includes collecting receiving air and wherein the system can further comprising a sample conditioner, and wherein the controller controls the conditioner to condition the received and wherein electronically sensing the air includes electronically sensing the conditioned air.

For example, the systems can further comprise an air collection device for collecting the sample of the air.

For example, the systems can further comprise an analyzer for identifying components present in the collected sample.

For example, the systems can further comprise an analyzer for effective for determining the chemical composition of the collected sample.

For example, the systems can further comprise an analyzer that comprises an olfactometer, a GC-MS (Gas Chromatography-Mass Spectroscopy), a SPME (Solid-Phase Micro-Extraction), a PFPD (Pulse-Flame Photometric Detectors), flame photometric detectors, flame ionization detector, a tandem mass spectrometry, gas chromatography-mass spectrometry—olfactory port, photoluminescence-based detector, Fourier transform infrared spectroscopy or combinations thereof.

For example, the controller can be effective for determining whether an event in the air is occurring based on at least the electronically sensing is carried out by means of a predetermined electronic sensory response and presence of such a predetermined electronic sensory response confirms occurrence of the event.

For example, the controller can be effective for electronically sensing the air; and determining whether an event in the air is occurring based on at least the electronically sensing is carried out by noting a change in the chemical composition of the air is detected or when a predetermined electronic sensory response or a predetermined electronic dimensional array signature (EDAS) is detected or not.

For example, the controller can be effective for electronically sensing the air; and determining whether an event in the air is occurring based on at least the electronically sensing is carried out by means of a predetermined electronic dimensional array signature and presence of such a predetermined electronic dimensional array signature in the air confirms occurrence of the event.

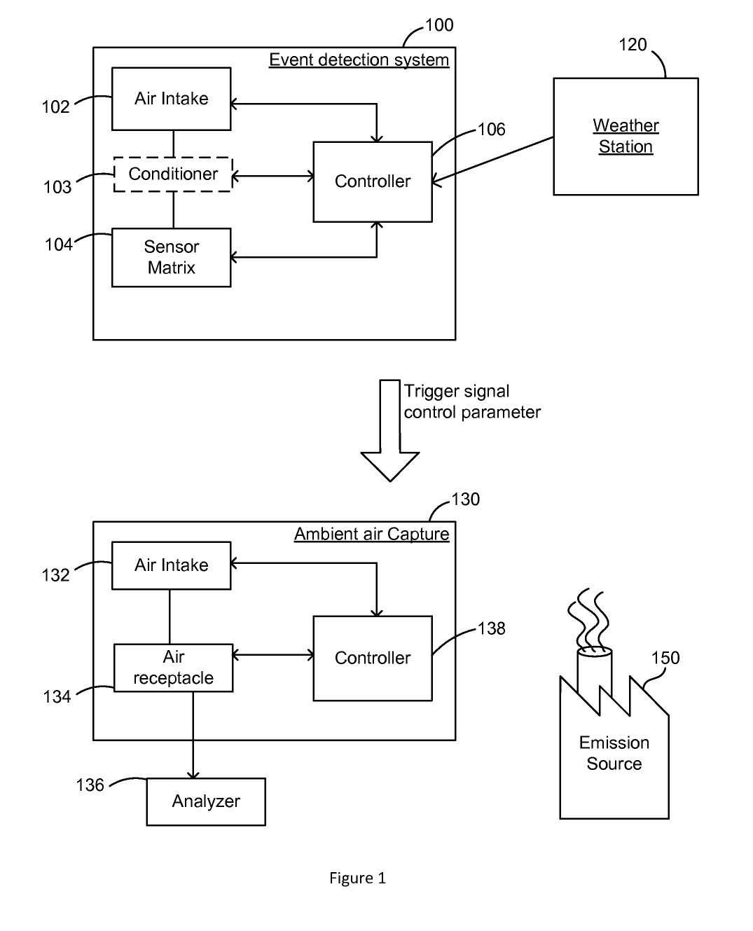

Referring now to FIG. 1, therein illustrated is a schematic diagram of an event detection system 100 in accordance with various exemplary embodiments. The event detection system 100 includes an air intake 102, at least one electronic sensor apparatus 104 and a controller 106. The event detection system 100 is capable of monitoring the air and detecting whether an event in the air is occurring.

The air intake 102 can receive air from the atmosphere. The air intake 102 is coupled to the electronic sensor apparatus 104 such that air received by the air intake 102 can be analyzed by the electronic sensor apparatus 104.

The electronic dimensional array signature or sensor apparatus 104 can electronically sense the air and output a result of the electronic sensing. For example, the electronic sensor apparatus 104 can provide a qualitative evaluation or unique electronic response qualitative of the air. Such characteristics can be, for example, the presence or absence of a given chemical or group of chemicals that generate specific electronic signature that can be recognized. The interaction of the sensors or EDAS can also be induced by the variations and composition of the respective chemical compounds proportion in the air. A qualitative evaluation can be a response or responses from sensors that allows for providing certain characteristics of the air. Such characteristics can be, for example, the presence or absence of a given chemical or family of chemicals that have a specific signature or fingerprint that can be recognized or identified by a sensor

For example, a chemical compound in the air has a unique EDAS. Two compounds present a new response multiple chemical compounds in the air generate yet a unique response. These EDAS responses can be part of the trigger mechanism to collect an air sample.

For example, the collective multi sensors can together provide an EDAS t unique signature to the chemical composition in the air. The response can be compared to a growing database and/or analyzed by a GC-MS to determine the group of chemicals inducing the characteristic signature.

For example, the electronic sensor apparatus 104 can detect the presence of certain odors within the ambient air. The electronic sensor apparatus 104 can measure an odor level. For example, the qualitative measurement simulates human olfaction. The electronic sensor apparatus 104 can provide a qualitative measurement of an ambient air within a short duration of time such that it appears that the measurement is provided substantially in real time. For example, the qualitative measurement can be provided by the EDAS. For example, the EDAS provides a “picture” of the odor as the human nose provides a “smell”.

According to various exemplary embodiment, the electronic sensor apparatus 104 can be a multi-sensor array based apparatus or an electronic nose. Where the electronic sensor apparatus 104 has a multi-sensor array of 2 or more sensors, at least one of the sensors is a non-specific sensor type or the sensor reacts to more than one chemical compound. For example, the sensors of the electronic sensor apparatus 104 can be MOS, quartz crystal microbalance, conducting polymer, microelectromechanical systems, surface acoustic waves and/or chemical cells.

There is no limit to the number of sensors in the collective, adding more sensors to the collective will further refine electronic dimensional array signature response.

One or more controllers described herein may be implemented in computer programs executing on programmable computers, each comprising at least one processor, a data storage system (including volatile and non-volatile memory and/or storage elements), at least one input device, and at least one output device. For example, and without limitation, the programmable computer may be a programmable logic unit, a mainframe computer, server, and personal computer, cloud based program or system, laptop, personal data assistance, cellular telephone, smartphone, or tablet device.

Each program is preferably implemented in a high level procedural or object oriented programming and/or scripting language to communicate with a computer system. However, the programs can be implemented in assembly or machine language, if desired.

In any case, the language may be a compiled or interpreted language. Each such computer program is preferably stored on a storage media or a device readable by a general or special purpose programmable computer for configuring and operating the computer when the storage media or device is read by the computer to perform the procedures described herein.

The controller 106 is operable to control the receiving of air by the air intake 102 and the sensing of the air by the electronic sensor apparatus 104. For example, the controller 106 is communicably connected to the air intake 102 to provide control signals to the air intake 102. For example, the controller 106 is also communicably connected to the electronic sensor apparatus 104 to provide control signals to the electronic sensor apparatus 104.

The controller 106 is further connected to the electronic sensor apparatus 104 to receive the result of the electronic sensing of the air from the electronic sensor apparatus 104. Where the electronic sensor apparatus 104 is a multi-array sensor apparatus, the result can include amplitude responses of the sensors. The result can also include amplitude variations of one or more of the sensors. The result can also include the relative response between the sensors. For example, the sensor responses are presented as a sensor response pattern or EDAS. For example, the sensor response can be a signature pattern or sensor fingerprints. Where the electronic sensor apparatus 104 is an electronic nose, the results can also be a qualitative reading.

According to various exemplary embodiments, the controller 106 can further receive meteorological data from a weather information source 120. For example, the weather information source 120 can be a weather station that monitors meteorological conditions in and around the geographical area where the event detection system 100 is located. Meteorological data can include temperature, humidity, pressure, wind direction, wind speed, and/or solar radiation.

According to various exemplary embodiments, the event detection system 100 can further have an air conditioner 103. The air conditioner 103 can treat a volume of the air received by the air intake 102 prior to the air being electronically sensed by the electronic sensor apparatus 104. Treatment of the air can improve the accuracy of the sensing carried out by the electronic sensor apparatus 104. For example, the air conditioner 103 can heat or cool volume of the air to a desired temperature. Additionally, or alternatively, the air conditioner 103 can adjust the humidity of the volume of the air to a desired humidity level, the filtration of particulates or other elements or substances.

Based on at least the result of electronic sensing, the controller 106 can further determine if an event is occurring in the air. An event represents a condition or change in condition of the air that suggests a significant change in the concentration levels of some gases in the air. For example, the concentration levels may have reached certain levels that can have a significant health or environmental impact. Accordingly, an event represents an interval of time at which a further analysis of the air should be conducted in order to more accurately measure the properties of the air. For example, an event in the air can be usually fluctuates in concentration of a given chemical in the air, a particular rate of change of at least one characteristic of the air, the modification of the composition of the air or at least one characteristic of the air having reached a particular threshold.

According to various exemplary embodiments, the determination of whether an event is occurring in the air is made based on at least the electronic sensing carried out by the electronic sensor apparatus 104. For example, an event may be reflected in the result of the electronic sensing as sensor amplitude variations, high or low sensor amplitude values, particular sensor patterns, signature pattern, or sensor fingerprints. The event detection system 100 can be appropriately configured according to its expected operating surroundings and the expected chemical properties to accurately identify the occurrence of an event from the result of the electronic sensing of the air.

According to one exemplary embodiment, the determination of whether an event is occurring is made by comparing the results of electronic sensing against a set of at least one predefined condition. For example, the set of at least one predefined condition can include a predetermined rate of change of sensor amplitudes, a high or low threshold value of sensor amplitudes, a predefined sensor pattern, signature pattern or sensor fingerprints. When the results of the electronic sensing satisfy the set of at least one predefined condition, it is determined that there is an indication of an event occurring. If the set of at least one predefined condition is not met, then an event is not occurring.

According to one exemplary embodiment, in addition to the determination of whether an event is occurring being based on the results of the electronic sensing, the controller also makes the determination based on at least one characteristic of the weather. The characteristic of the weather can be obtained from the received meteorological data.

Alternatively, or additionally, the determination can be further based on past or forecasted meteorological data.

According to various exemplary embodiments, the electronic sensing of the air by the electronic sensor apparatus 104 and the determination of whether an event is occurring can be carried out substantially in real time. For example, the controller 106 can control the air intake 102 and the electronic sensor apparatus 104 such that their respective steps are carried out one after another within a short duration of time. Furthermore, the controller 106 can determine whether an event is occurring immediately after the result of the electronic sensing is received. By carrying out all of the steps within a short duration of time, the actions appear to be taken in real time.

According to various exemplary embodiments, the event detection system 100 can monitor the air by repeatedly receiving air at the air intake, electronically sensing the collected sample using the electronic sensor apparatus 104 and determining whether an event is occurring. The event detection system can monitor the air in a substantially continuous manner. It will be understood that the substantially continuous monitoring is the result of repeating the steps at short time intervals apart such that a plurality of discrete measurements closely spaced in time are made. As a result, the monitoring appears to be continuous to a human operator. Moreover, where the steps are repeated and carried out in real-time, the substantially continuous monitoring appears to be in real-time.

According to various exemplary embodiments, the event detection system 100 can continuously monitor the air. The continuous monitor can further be a continuous qualitative monitoring.

Where the event detection system 100 monitors the air over time by repeating the steps, the controller 106 can be further configured to store a log of the results of the electronic sensing and/or of determinations of whether an event is occurring. For example in addition to, or as an alternative to, determining whether an event is occurring based on the received meteorological data, the determination can be based on previously logged results or determinations.

The controller 106 can further trigger other components of the event detection system 100 or trigger an external device, such as an apparatus for conducting a further analysis of the air. When the controller 106 determines that an event in the air is occurring, the controller 106 triggers the collecting a sample of the air for a further analysis.

According to various exemplary embodiments, the controller 106 can further monitor whether an event is ongoing. A first determination that an event is occurring in the air indicates that an event has begun. Then in subsequent repetitions of the steps of monitoring the air, further determinations that an event is occurring indicates that the event is ongoing. A determination that an event is not occurring indicates that an event has ended. For example, controller 106 can be configured to trigger the end of the collecting of a sample for further analysis when a determination has been made that the event has ended.

For example, according to exemplary embodiments where the determination of whether an event is occurring is based on whether the result of the electronic sensing satisfy a set of at least one predefined condition, if the set of condition is met in subsequent repetitions, then it is determined that the event is ongoing. Where the condition for an event is no longer met, it is determined that the event has ended. Alternatively, where the condition for an event is no longer met for a predefined amount of time or predetermined number of repetitions of the steps of monitoring the air, it is determined that the event has ended.

According to various exemplary embodiments, in addition to triggering the beginning of a further analysis, the controller 106 can further provide various control parameters for configuring the further analysis to be carried out. For example, the control parameters are for adjusting the collecting of a further sample of the air for further analysis. For example, the control parameters can be based on the different results of the electronic sensing. For example, the control parameters are selected based on a comparison to predefined rate of change of sensor amplitudes, high or low threshold value of sensor amplitudes, sensor pattern, signature pattern or sensor fingerprints. The control parameters can be further based on the meteorological data received from the weather station 120.

According to one exemplary embodiment, the result of the electronic sensing and optionally the meteorological data are provided as the control parameters. Alternatively, the controller 106 calculates based on the qualitative measurement and meteorological data appropriate control parameters to be provided. For example, where the control parameters are sent to an external device that can only receive specific types of inputs, the controller 106 computes and/or converts the result of the electronic sensing and meteorological data to the suitable inputs.

For example, the control parameter can indicate the type of the further analysis, the duration, the volume of gas to be collected, and/or specific types of gases to measure. For example, the control parameters can include the time, period, frequency, temperature, or level of humidity, barometric pressure as part of the criteria to trigger the logical control to initiate sample collection.

The methods and systems of the presen disclosure can also be installed over complex terrain where a sample would be collected based on all of the above mentions and relative to other units before initiating sample collection in one or more remote units to capture the response during unique wind conditions where wind direction and speed is modified by the terrain it flows over.

The controller 106 may implemented as plurality of controllers operating together. For example, a first controller, which may be a microcontroller, controls the air intake and sensor matrix of the event detection system in order to sense air. A second controller, which may be a programmable computer, receives results from the electronic sensor apparatus 104 and determines based on at least the received results whether an event is occurring. The second controller can further determine the control parameters.

Continuing with FIG. 1, the event detection system 100 can be connected with a air capture device 130. Preferably, the air capture device 130 is located proximate the event detection system 100. The event detection system 100 can be retrofitted to an air capture device 130 that has already been deployed.

The air capture device 130 can comprise an analyzer 136 or be connected to an analyzer 136. The analyzer 136 can be a non-continuous analyzer having a lower detection threshold and capable of measuring a large range of chemicals. For example, the analyzer 136 can be a gas chromatograph, such as a gas chromatography-mass spectrometry, solid-phase micro-extraction pulse-flame photometric detectors, flame photometric detectors, flame ionization detector, a tandem mass spectrometry, gas chromatography-mass spectrometry—olfactory port, Photoluminescence-based detector, Fourier transform infrared spectroscopy. The analyzer 136 can be an analyzer already deployed in the field for measuring characteristics of the air.

The event detection system 100 triggers the air capture device 130 to begin collecting a sample of the air for further analysis. For example, the controller 106 sends a trigger signal, which may be an electronic signal, to the air capture device 130. When the trigger signal is received, the air capture device 130 begins the collection of the sample of the air for further analysis.

As illustrated in FIG. 1, the air capture device 130 has an air intake 132, air receptacle 134, and controller 138. The air intake 132 of the air capture device 130 can collect at least one sample of the air from the atmosphere.

The sample of air collected by the air intake 132 is stored in one or more air receptacles 134. For example, the air intake 132 can be a variable pump that has a variable intake rate to accommodate the type of sampling and sampling parameter. For example, sampling parameters include duration, flow rate, barometric pressure, period frequency, temperature, humidity level. For example, the receptacles 134 can be containers such as cartridges, canisters, and/or bags (such as Tedlar™, Teflon™ or Nalophan™ bags). The volume of air collected in the air receptacles can be measured and/or monitored.

The analyzer 136 receives the collected sample of air and performs the further analysis of the air to determine properties of the air. According to some exemplary embodiments, the analyzer 136 is part of the air capture device 130 and can receive the collected sample of the air. Alternatively, as shown, the analysis equipment can be located remotely, and the samples of the air collected in the one or more air receptacles 134 can be transported to the location of the analyzer 136 for further analysis. For example, the analyzer 136 can carry out an analysis of the sample to identify major components present in the sample. For example, the analyzer 136 can carry out a chemical analysis of the sample to identify components present in the sample. For example, the analyzer 136 can carry out a chemical analysis of the sample to identify at least one component present in the sample. For example the analysis equipment can be a non-continuous gas analyzer.

The controller 138 is operable to control the collecting of samples of the air by the air intake 132, the receiving of those samples within the one or more air receptacles.

According to some exemplary embodiments, the controller 138 can further control the determination of properties of the air by the analyzer 136.

The trigger signal sent from the controller 106 of the event detection system 100 is received by the controller 138 of the air capture device 130. The controller 106 then controls the air intake 132 and air receptacles 134 to carry out the collection of at least one sample of the air for further analysis. According to exemplary embodiments where control parameters are also sent, these parameters are received by the controller 138, and control of components of the air capture device 130 is carried out according to the received control parameters.

According to one exemplary embodiment, the collection of the sample of the air for further analysis is carried out for a predetermined duration.

According to another exemplary embodiment, the duration, flow rate, period, and/or of the collection of the sample of the air for further analysis is specified as one of the control parameters, and the collection of the sample is carried out according to the received control parameter.

According to yet another exemplary embodiment, the duration of the collection of the sample for further analysis is defined by the trigger signal to begin the collection and the trigger signal to end the collection. Accordingly, the collection of the further sample is started when the controller 138 receives the trigger signal from the controller 106 of the event detection system 100 to begin the collection. The collecting of samples of the air in the air receptacles 134 is continued so long as the trigger signal to end the collection is not received, or the air receptacles 134 have not become full. When an end trigger signal is received or the air receptacles 134 become full, the collecting of samples of the air is stopped. An analysis of the collected sample can then be carried out by the analyzer 136. Alternatively, the collected sample can be stored for further analysis at a later time.

It will be appreciated that this method of the collection of the sample of the air for further analysis according to the latter exemplary embodiment provides for correlation between duration of the collection of the air and the duration of the detected event of the air. In particular, after an event is detected, the collection is carried out while the event is ongoing. When an end to the event is detected, the collection is also ended. Advantageously, this provides for more accurate measurements of conditions of the air by allowing analysis of the entirety of the event. In comparison, where the collection is not correlated with the duration of the detected event, it is possible that the collection of the air will be carried out for only a portion of the event, or for a longer duration than the event itself.

The controller 138 of the air capture device 130 may be implemented as a plurality of controllers operating together. For example, a first controller, which may be a microcontroller, controls the air intake 132 and monitors the volume of the air receptacles 134. A second controller, which may be a programmable computer, receives outputs of the electronic sensor apparatus 104 and determines based on at least the received outputs whether there is an event.

Whereas the event detection system 100 only makes a qualitative measurement of the collected air sample, the air capture device 130 can perform a further analysis. Advantageously, the qualitative measurements can be carried to monitor the air in a continuous manner and substantially in real time. Accordingly, the event detection system 100 provides a lower probability that some events will be missed. In triggering a collecting of a sample of the air for further analysis when an event is detected, the sample of the air is collected when a state of the air requires it. Therefore a more targeted approach is taken to analyzing the air. Given that a gas chromatograph can often be expensive and time consuming, such a targeted approach provides more efficiency and cost savings.

Continuing with FIG. 1, the event detection system 100 and air capture device 130 can be deployed to monitor concentration levels of certain gases being emitted from an emission source 150. For example, the emission source 150 can be an industrial operation or landfill or other industrial, commercial or residential, municipal setting. It can also come from airports, port, petrochemical industry or mining or oil and gas operations. Such sources are known for potentially emitting harmful gases. The emitted gases are dispersed over the atmosphere and can reach areas that can be affected by the gases. The event detection system 100 and air capture device 130 are deployed within the area of dispersion of the emitted gases to monitor their levels.

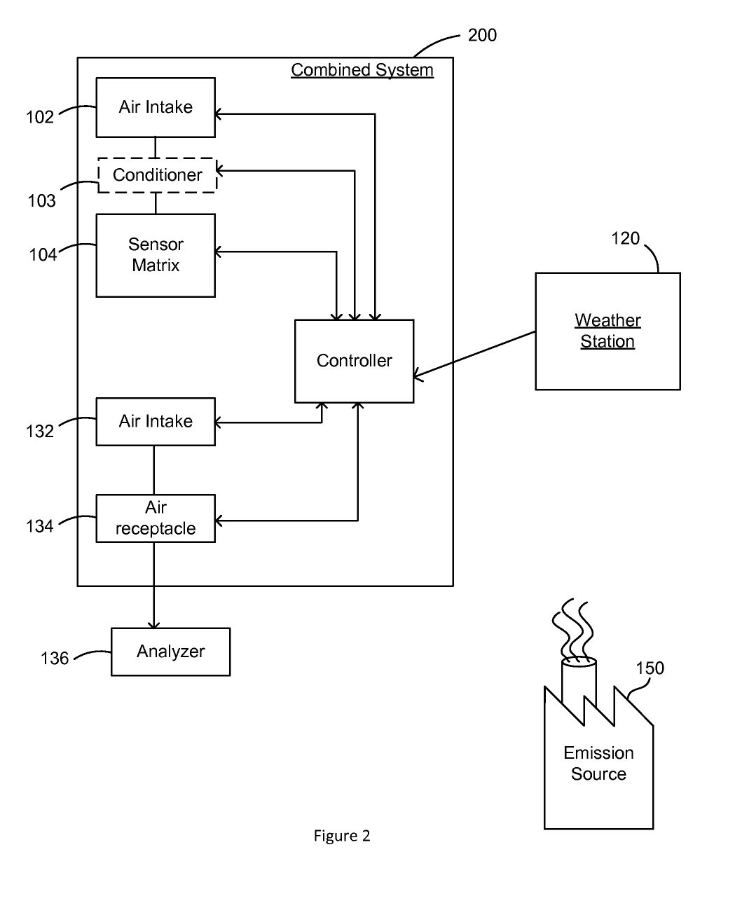

Referring now to FIG. 2, therein illustrated is a schematic diagram a combined event detection and air capture system 200 according to on alternative embodiment. The combined event detection and air capture system 200 has the components of the event detection system 100 and air capture device 130 as described herein with reference to FIG. 1. However, the event detection system 100 and air capture device 130 have been merged into a single system. For example, a controller 202 controls the air intake 102, electronic sensor apparatus 104, air intake 132, air receptacle 134. The controller 202 can further control the analyzer 136. However, it will be understood that controller 202 is shown as a single controller for ease of illustration only, and that according to various exemplary embodiments, the controller 202 can be implemented as a plurality of controllers operating together. Advantageously, the combined event detection and air capture system 200 provides a ready-to-use solution for event-based detection and analysis of the air.

Unlike the event detection system 100 of FIG. 1, the controller 202 of the combined event detection and air capture system 200 does not need to send trigger signals to an external device to carry out the collection of a sample of the air for further analysis. Instead, the controller 202 triggers a collecting of a sample for further analysis by sending commands to the air intake 132 and air receptacle 134 of the combined system 200.

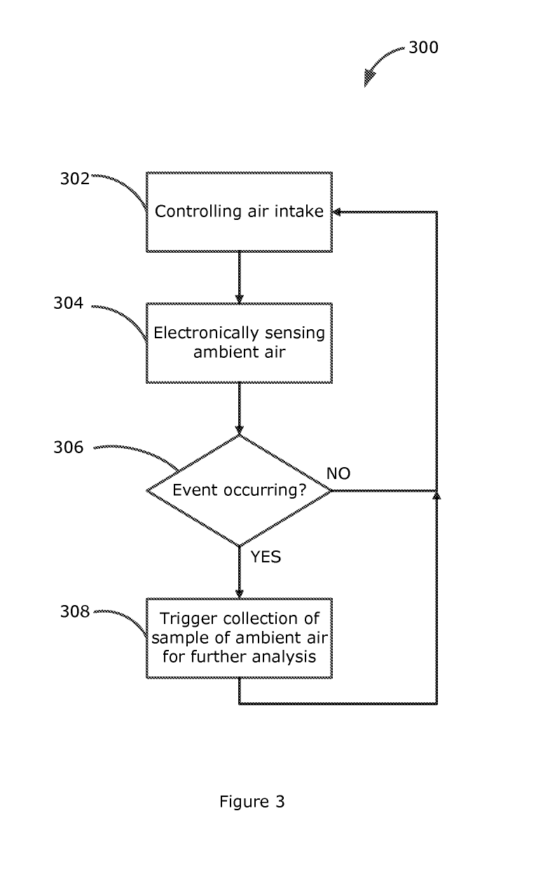

Referring now to FIG. 3, therein illustrated is a schematic diagram of a method 300 for monitoring the air.

At step 302, the air intake is controlled to receive air. For example, the controller 106 can send a control signal to the air intake 102 of the event detection system 100 to open a valve of the air intake 102. The controller 106 can further control the air intake 102 to close the valve after a duration of time.

At step 304, the air is electronically sensed. For example, the electronic sensing is carried out by the electronic sensor apparatus 104. For example, the result generated from the electronic sensing is a qualitative measurement of the sample.

According to various exemplary embodiments, prior to electronically sensing the air at step 304, the received volume of air can be conditioned. Conditioning the received can improve the accuracy of the sensing at step 304. For example, the conditioning of the sample is carried out by the air conditioner 103.

At step 306, it is determined whether an event is occurring in the air. For example, it can be determined that an event is occurring if the results of the electronic sensing at step 304 satisfy a set of at least one predefined condition. The determination of whether an event is occurring can be further based on meteorological data indicating at least one weather condition. Additionally, or alternatively, the determination of whether an event is occurring can be further based on at least one previous result of electronic sensing. For example, the determination of whether an event is occurring can be carried out by the controller 106 of the event detection system 100.

If it is determined at step 306 that an event is not occurring in the air, the method proceeds to step 302 to continue monitoring the air for an event.

If it is determined at step 306 that an event is occurring in the air, the method proceeds to step 308 to trigger the start of the collecting of a sample of the air for further analysis. The method then proceeds to step 302 to continue monitoring the air for an event.

According to various exemplary embodiments, steps 302, 304 and 306 are carried out within a short duration of time. Preferably, the steps are carried out substantially in real time. Furthermore, if it is determined at step 306 that an event is occurring, step 308 can also be carried out within a short duration of time. Preferably, step 308 is also carried out in real time along with steps 302, 304, and 306.

According to various exemplary embodiments, steps 302, 304, and 306 are repeated at short times intervals apart such that the monitoring of the at least one condition of the air is substantially continuous.

According to various exemplary embodiments, the event detection system 100 can continuously monitor the air. The continuous monitoring can further be a continuous qualitative monitoring.

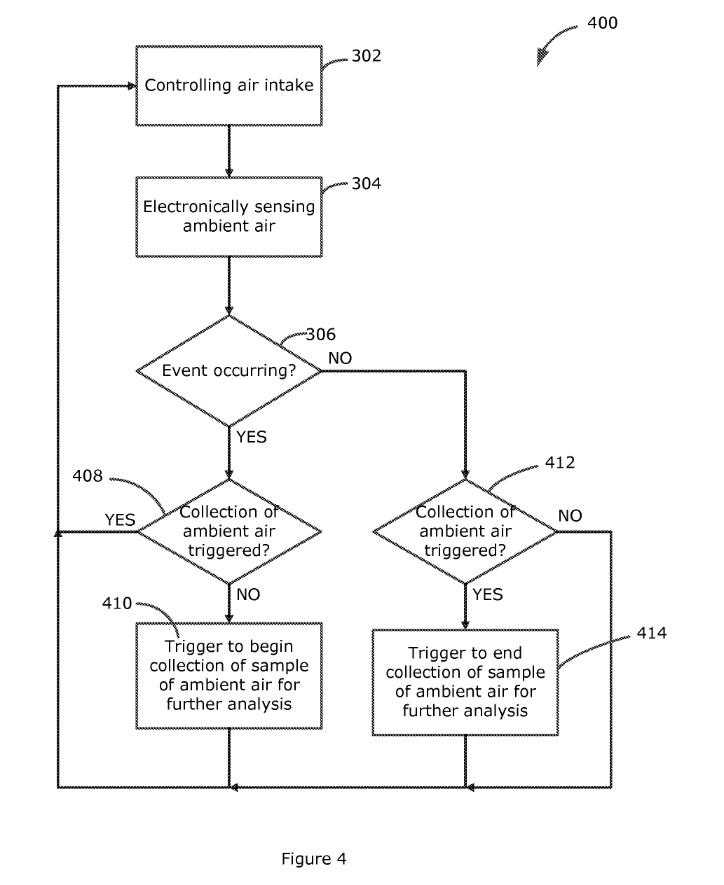

Referring now to FIG. 4, therein illustrated is a schematic diagram according to an alternative exemplary method 400 for monitoring the air. The step 302 (controlling the air intake to collected at least one sample), step 304 (electronically sensing the collected sample), and step 306 (determining whether an event is occurring in the air) are still carried out to monitor the air.

If it is determined at step 306 that an event is occurring, the method proceeds to step 408 to determine whether a start of a collection of a sample of the air for further analysis has already been triggered.

If it is determined at step 408 that a collection of the sample for further analysis has not been triggered, then the method proceeds to step 410 to trigger the start of a collecting of a sample of the air for further analysis. The method then proceeds back to step 302 to continue monitoring the air for an event.

A determination at step 408 that a collecting of a sample for further analysis has already been triggered indicates that the event in ongoing. The method proceeds back to step 302 to continue monitoring the air for an event.

If it is determined at step 306 that an event is not occurring, the method proceeds to step 412 to determine whether a start of a collecting of a sample for further analysis has already been triggered.

If it is determined at step 412 that a collecting of a sample for further analysis has not been triggered, the method proceeds back to step 302 to continue monitoring the air for an event.

A determination at step 412 that a collecting of a sample for further analysis has already been triggered indicates that the event was previously ongoing and that a collecting of a sample for further analysis is still ongoing. However, a determination that the event is not occurring indicates that the event has come to an end. The method proceeds to step 414 to trigger an end of the collecting of the sample for further analysis. The method then proceeds back to step 302 to continue monitoring the air for an event.

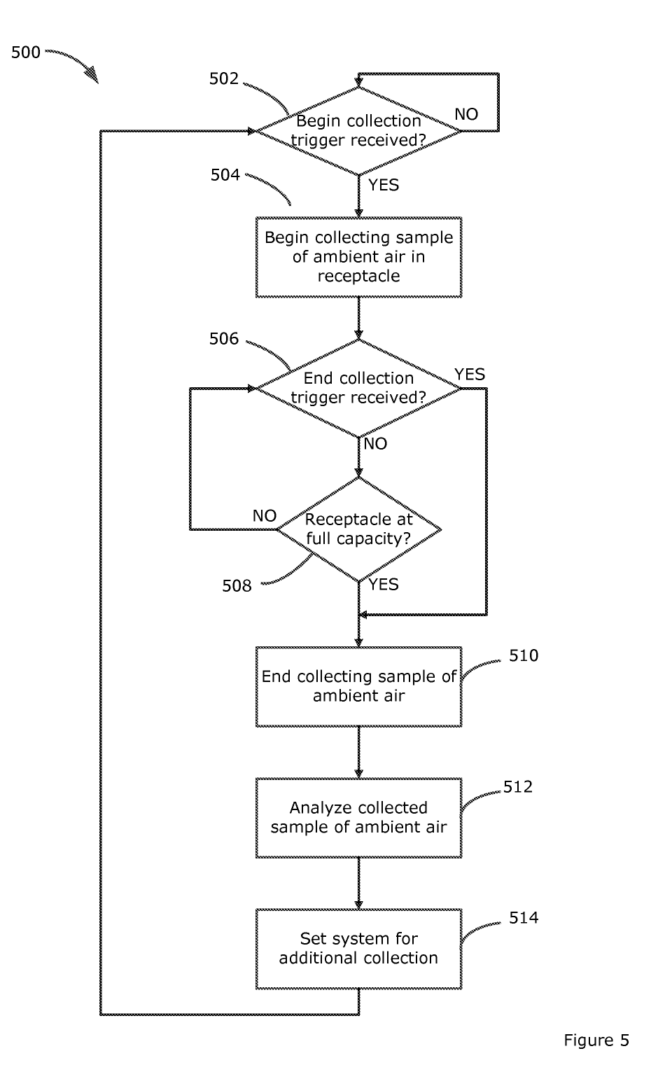

Referring now to FIG. 5, therein illustrated is an exemplary method 500 for carrying out a collecting of a sample of the air for further analysis according to various exemplary embodiments. The collecting of the sample according to method 500 includes correlating the duration of the collecting of the sample of the air for further analysis with the duration of the detected event of the air.

At step 502, it is determined whether a trigger to begin the collecting of the sample for further analysis has been received. When the trigger to begin the collecting has been received, the method proceeds to step 504. For example, the controller 138 receives trigger signals and control parameters from controller 106 of the event detection system 100.

According to various exemplary embodiments, the collecting of the sample for further analysis is not begun immediately after receiving a first trigger. For example, the first trigger may be the result of a false indication of an event in the air. Instead, after receiving a first trigger, there may be a waiting time interval before determining whether a second trigger has been received. Providing a waiting interval and listening for at least two triggers reduces the probability that a collecting of the sample for further analysis is started in response to a false indication of an event. Alternatively, the collecting of the sample for further analysis can be started only after receiving a specified amount of consecutive triggers or a specified amount of triggers within a predefined time interval.

At step 504, the collecting of the sample for further analysis is started. For example, an air intake 132 of the air capture device 130 is controlled to open a valve of the air intake 132. The air flowing through the air intake 132 is then collected in the one or more air receptacles 134.

At step 506, it is determined whether a trigger to end the collecting of the sample for further analysis has been received. If a trigger to end the collecting of the sample for further analysis has been received, the method proceeds to step 510. If a trigger to end has not been received, the method proceeds to step 508.

According to various exemplary embodiments, the collecting of the sample for further analysis is not ended immediately after receiving a first trigger to end the collecting. For example, the first trigger to end may be result of a false indication of the end to an event in the air. Instead, after receiving a first trigger to end, there may be a waiting time interval before determining whether a second trigger end has been received. Providing a waiting interval and listening for at least two triggers reduces the probability that a collecting of the sample for further analysis is ended in response to a false indication of an event being ended. Alternatively, a collecting of the sample for further analysis can be ended only after receiving a specified amount of consecutive triggers or a specified amount of triggers within a defined time interval.

At step 508, it is determined whether the one or more air receptacles have reached full capacity. If the air receptacle has not reached full capacity, the method returns to step 506 to listen for a trigger to end the collecting of the sample for further analysis. It will be appreciated that where a trigger to end the collecting of the sample for further analysis is not received at step 506 and the receptacle has not reached full capacity 508, the collecting of the sample of air is continued.

If the air receptacles have reached full capacity at step 508, the method proceeds to step 510.

At step 510, the collection of the sample of the air for further analysis is ended. For example, the valve of the air intake 132 is controlled to stop collecting the sample of the air in the air receptacles.

The collected additional sample of the air is optionally analyzed at step 512. For example, the analysis can be carried out by the analyzer 136 of air capture device 130. For example, the collected sample can be immediately analyzed at step 512. Alternatively the collected sample can be stored, and the further analysis is carried out at a later time. For example, the analyzer 136 may be located remotely, and the collected sample is transported to be analyzed by the analysis equipment. For example the analysis can be a gas chromatography. For example, the analyzer 136 can carry out an analysis of the sample to identify major components present in the sample. For example, the analyzer 136 can carry out a chemical analysis of the sample to identify components present in the sample. For example, the analyzer 136 can carry out a chemical analysis of the sample to identify at least one component present in the sample.

At step 514, the air capture device 130 is set so that an additional collection of a sample of the air for further analysis can be carried out. The method then proceeds back to step 502 to listen for an additional trigger to begin another collecting of a sample of the air for further analysis.

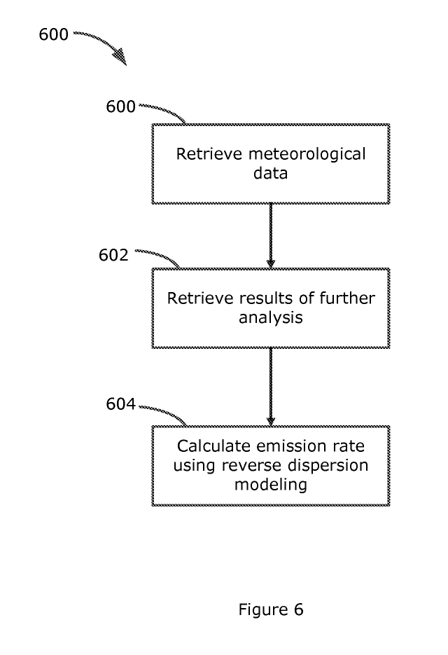

Referring now to FIG. 6, therein illustrated is a schematic diagram of a method 600 according to various exemplary embodiments for calculating the emission rate at the source of the emission based on concentration levels of gases measured from the further analysis of the collected sample of the air. For example, the method 600 can be carried out by any suitable computer system operable to receive meteorological data and results of a further analysis.

At step 602, meteorological data is retrieved. The meteorological data can correspond to the time at which the collecting of the sample for further analysis was carried out. Where the further analysis was carried out for the duration of an event, the meteorological data for substantially the same duration is retrieved. The meteorological data retrieved can also correspond to the geographical area where both the emission source 150 and the air capture device 130 carrying out the collecting of the sample for further analysis are located.

At step 604, the results of the further analysis carried out by the analyzer 136 are retrieved. In particular, the measurements of the concentration levels of specified gases obtained from the further analysis are retrieved. The specified gases can correspond to those gases that are expected to be emitted by the emission source.

At step 606, reverse dispersion modeling is applied to calculate a predicted emission rate at the emission source. The retrieved meteorological data, the concentration levels of the specified gases, the location of the emission source 150 and the location of the air capture device 130 are used as inputs for the reverse dispersion modeling.

The result of the reverse dispersion modeling at step 606 is a predicted emission rate at the emission source. For example, the predicted emission rate can be useful for monitoring whether the emission source 150 is complying with specified emission limits. For example, the emission limits can be set out in laws or regulations that apply to the emission source 150.

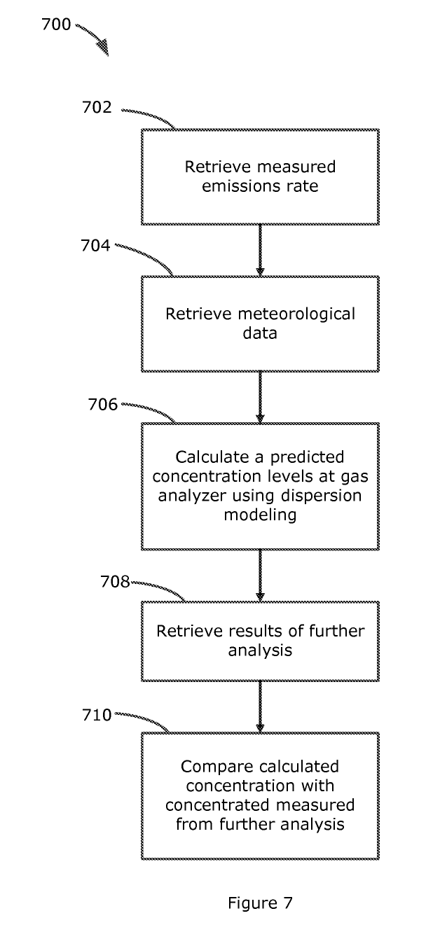

Referring now to FIG. 7, therein illustrated is a schematic diagram of a method 700 according to various exemplary embodiments for validating dispersion modeling used to predict concentration levels of specified gases at a location remote of the emission source 150. For example, the method 600 can be carried out by any suitable computer system. An event detection system 100 and air capture device 130 are positioned at the remote location to monitor and measure conditions of the air at the remote location.

At step 702, meteorological data is retrieved. The meteorological data can correspond to the time at which the collection of the sample for further analysis was carried out. Where the collection was carried out for the duration of an event, the meteorological data for the same duration is retrieved. The meteorological data retrieved can also correspond to the geographical area where both the emission source 150 and the air capture device 130 carrying out the collection are located.

At step 704, the emissions rate of specified gases at the emission source 150 are retrieved. The specified gases can correspond to those gases emitted by the emission source 150 that can potentially have a significant environmental impact.

At step 706, dispersion modeling is applied to calculate predicted concentration levels of the specified gases at the remote location. The retrieved meteorological data, the emissions rate at the emission source, the location of emission source 150 and the location of the air capture device are used as inputs for the dispersion modeling.

The result of the dispersion modeling is predicted concentration levels of the specified gases at the remote location

At step 708, the results of the further analysis carried out by the analyzer 136 are retrieved. In particular, the measurements of the concentration levels of specified gases obtained from the further analysis are retrieved.

At step 710, the measured concentration levels are compared with the predicted concentration levels to validate the results of the dispersion modeling.

Predicting concentration levels and validating the predictions can be useful for the emission source to control its emissions rate. In particular, the emission source 150 can control its emissions rate such that the concentration levels of the specified gases at the remote location is kept within acceptable limits. For example, the emission limits can be set out in laws or regulations that apply to the emission source 150. For example, an industrial or mining operation can control its emissions to keep concentrations level at a sufficiently low level at a nearby residential area or nature preserve.

It was found that by using the methods and the systems of the present disclosure, it was possible to continuously monitor air quality using a setup that is less expensive than current continuous air quality analyzers. The continuous monitoring can be for a greater number of chemicals and have a lower detection threshold. Based on the continuous monitoring, it was possible to identify events in the air that can represent the presence of poor air quality. It was also possible to trigger a non-continuous analysis of the air when an event representing poor air quality is detected. It was further possible to use non-continuous analyzers in a more targeted and efficient manner. These non-continuous analyzers have lower detection thresholds and respond to large range of chemicals.

It was also found that by using the methods and systems of the present disclosure, it was possible to use of a continuous (and optionally) non-specific chemical detection approach to identify changes in the chemical characteristic or composition of the air in conjunction or without metrological parameters, to trigger sampling for further analysis or characterization. It was also possible to identify specific poor air quality Events to get more detailed characterization out of continuously changing conditions that would provide not otherwise be detected using traditional air monitoring equipment. Such methods and systems were efficient for carrying out an automation of snapshot air quality sampling at relevant conditions previously performed manually without clear triggering parameters. It was thus possible to have an automation based on conditions set by machine measurement for improved relevance, higher precision and frequency.

The systems and methods of the present disclosure allow for a lower-cost, higher efficiency cycle of chemical or olfactometry analysis as well as reducing potential errors caused by mishandling of the samples and improvement in quality assurance and quality control of samples. These systems and methods can allow for large spectrum of chemical detection. They can be seen as inexpensive tools to obtain detailed information of complex air quality issues or events during their occurrence.

The previous technologies of the prior art are mainly using a GCMS to sample the air for short periods then are offline while the sample is processed; missing the event during the processing time. The systems and methods of the present disclosure are effective for overcoming such a drawback by sensing the air and taking samples only when an event has occurred, thereby significantly increasing the probability of identifying the chemicals composition resulting in the event.

The systems and the methods of the present disclosure can have the flexibility to focus on different combinations of factors that could generate an event and they have the capacity to assess the individual and cumulative contribution of multiple sources.

The systems and the methods of the present disclosure can also capture the occurrence of second generation pollutants caused by the interaction of the chemicals from various sources and meteorological conditions.

The systems and the methods of the present disclosure can have the capacity of instantaneously capturing air samples for detailed chemical characterization in the occurrence of an event. For example, they can capture an event for chemical characterization and thereby allowing the attribution to a source(s) using dispersion modeling, atmospheric chemistry changes and local meteorological data, and calculate the emission rates. For example, the systems and methods of the present disclosure can sample only for the duration of the event and eventually to stop to stop sampling if the event has come to an end.

It was found that in prior art that in a complex terrain, a meteorological tower may not be representative of ground wind effects. Especially in a valley / hilly terrain An example of this is in a valley or rough terrain setting where soft winds flow in one direction and are redirected by terrain. The systems and methods of the present disclosure can be deployed to help track, monitoring and sample air quality even under such difficult conditions.

It was found that the systems and methods of the present disclosure can be quite useful for example when using the enhanced electronic noses and vector monitoring to differentiate emissions from individual facilities. Using the installed wind senor the computer can be programmed to only sample air when it is blowing only from a certain vector giving it the ability to selectively sample the air from a source.

It was also found that the systems and methods of the present disclosure can be useful to source monitoring and/or to monitor air contaminants coming of or entering a project area. i.e. being able to differential emissions from adjacent operations.

The systems and methods of the present disclosure can represent an inexpensive alternative to a full station and they can be integrated equipment as needed to identify specific contaminants once identified. The existing issue with all WBEA data is it does not tell where the main source is or how much one company is contributing. This is focus on one operation or source and only sample when parameters are within predefined specifications. Such problems can be clearly overcome by using the systems and methods of the present application.

While the above description provides examples of the embodiments, it will be appreciated that some features and/or functions of the described embodiments are susceptible to modification without departing from the spirit and principles of operation of the described embodiments. Accordingly, what has been described above has been intended to be illustrative and non-limiting and it will be understood by persons skilled in the art that other variants and modifications may be made without departing from the scope of the disclosure as defined in the claims appended hereto.

1. A method for analyzing air, the method comprising:
monitoring the air, the monitoring comprising:
electronically sensing the air; and
determining whether an event in the air is occurring based on at least the electronically sensing; and
if an event is occurring, collecting a sample of the air for a further analysis.

2. The method of claim 1, further comprising carrying out the further analysis of the sample for identifying at least the major components present in the sample.

3. The method of claim 1, further comprising carrying out the further analysis, wherein the further analysis is a chemical analysis of the sample for identifying components present in the sample.

4. The method of claim 1, further comprising carrying out the further analysis, wherein the further analysis is a chemical analysis of the sample for identifying at least one component present in the sample.

5. (canceled)

6. The method of claim 1, wherein the further analysis is carried out by means of an olfactometer, a GC-MS (Gas Chromatography-Mass Spectroscopy), a SPME (Solid-Phase Micro-Extraction), a PFPD (Pulse-Flame Photometric Detectors), flame photometric detectors, flame ionization detector, a tandem mass spectrometry, gas chromatography-mass spectrometry—olfactory port, Photoluminescence-based detector, fourier transform infrared spectroscopy or combinations thereof.

7. (canceled)

8. The method of claim 1, wherein the monitoring the air is a continuous qualitative monitoring.

9. The method of claim 8, wherein the electronic sensing is a qualitative evaluation of the air.

10. The method of claim 9, wherein the electronically sensing of the air and the determination of whether an event is occurring are carried out substantially in real time.

11. The method of claim 10, wherein the electronically sensing of the air and the determination of whether an event is occurring are repeated at short time intervals apart, and wherein the monitoring of the air is substantially continuous.

12. The method of claim 11, wherein a result obtained from the electronically sensing of the air is compared to a predefined sensor reaction pattern, a signature pattern or sensor fingerprints in order to determine if an event is occurring.

13. The method of claim 12, wherein the monitoring further comprises conditioning a volume of the air to improve sensing accuracy and wherein electronically sensing the air includes sensing the conditioned volume of the air.

14. (canceled)

15. The method of claim 11, wherein the determination of whether an event is occurring is further based on at least one of:
at least one weather characteristic of the air; and
at least one previous result of the electronic sensing.

16. (canceled)

17. The method of claim 1, wherein collecting the sample for further analysis is made in accordance with parameters that are adjusted in view of the result obtained from the electronically sensing of the air that is compared to a predefined sensor reaction pattern, a signature pattern or sensor fingerprints.

18. (canceled)

19. The method of claim 17, wherein determining whether an event in the air is occurring is based on whether a result of the electronic sensing satisfies at least one predefined condition.

20-22. (canceled)

23. The method of claim 19, wherein the further analysis of the sample of air comprises conducting a gas chromatography of the air.

24. The method of claim 1, further comprising:
after triggering the start of the collecting of the sample of the air, if the event is no longer occurring, triggering an end of the collecting of the sample of the air.

25-28. (canceled)

29. A system for analyzing air, the system comprising:
an air intake;
an electronic sensor;
a controller configured for:
monitoring the air, the monitoring comprising:
controlling the air intake;
controlling the electronic sensor to electronically sense the air; and
determining whether an event in the air is occurring based on at least the electronically sensing; and
if an event is occurring, collecting a sample of the air for a further analysis.

30-32. (canceled)

33. The system of claim 29, wherein the electronic sensor comprises a multi-sensor array, wherein at least one of the sensor of the apparatus is non-specific.

34-37. (canceled)

38. The system of claim 29, further comprising an analyzer that comprises an olfactometer, a GC-MS (Gas Chromatography-Mass Spectroscopy), a SPME (Solid-Phase Micro-Extraction), a PFPD (Pulse-Flame Photometric Detectors), flame photometric detectors, flame ionization detector, a tandem mass spectrometry, gas chromatography-mass spectrometry—olfactory port, photoluminescence-based detector, Fourier transform infrared spectroscopy or combinations thereof.

39. The system of claim 38, wherein said controller is effective for determining whether an event in the air is occurring based on at least the electronically sensing is carried out by means of a predetermined electronic sensory response and presence of such a predetermined electronic sensory response confirms occurrence of said event.

40-41. (canceled)
